(19) United States
(12) Patent Application Publication
(10) Pub. No.: US 2008/0045418 A1
(43) Pub. Date: Feb. 21, 2008

(54) METHOD OF LABELING AND PROFILING RNAS

(76) Inventor: Xueliang James Xia, Chandler, AZ (US)

Correspondence Address: THE LUTHER LAW FIRM, 12198 E. COLUMBINE DR., SCOTTSDALE, AZ, 85259, US

(21) Appl. No.: 11/834,605

(22) Filed: Aug. 6, 2007

Related U.S. Application Data
(63) Continuation-in-part of application No. 11/116,935, filed on Apr. 28, 2005.
(60) Provisional application No. 60/650,034, filed on Feb. 4, 2005.

(51) Int. Cl. C40B 30/04; C12N 9/00

(52) U.S. Cl. 506/9; 435/183

(57) ABSTRACT

Disclosed herein is a method of selectively labeling non-messenger RNA molecules by isolating total RNA from a tissue or cell, dissolving the isolated RNA, optionally blocking 3′ ends of the RNA, and adding T4 RNA ligase and a labeled nucleic acid adaptor, with the result that the T4 RNA ligase ligates the adaptor to RNA having a 5′ phosphate group such as small RNAs. A method of labeling the 5′ end of mRNA isolates total RNA from a tissue or cell; dissolves RNA in RNase-free water; removes a 5′ cap structure from the mRNA using tobacco acid pyrophosphatase (TAP); removes the TAP; optionally blocks the 3′ end of the RNA molecules using TdT with ddA, ddT, ddG, ddC or a combination thereof; and ligates an adaptor to the RNA by adding T4 RNA ligase and a labeled DNA or RNA adaptor. A method of amplifying and labeling noncapped RNAs and/or capped RNAs is useful in expression analysis of the whole-genome transcripts from cells and tissues. Sense and antisense transcripts are labeled from different experimental approaches. In another embodiment, there is disclosed a method of sequence selection and probe design. Probes are designed complementary to the labeled target RNA. For small RNAs, sequences are selected for detections of mature, counterparts (ig miR*), and precursors. In another embodiment, there is disclosed a method of expression profiling small RNA by providing labeled small RNA, providing a microarray comprising a plurality of probes hybridizable to small RNA, incubating the labeled small RNA with the microarray, washing unhybridized RNA from the microarray and drying the microarray, staining hybridized RNA on the microarray; and scanning the labeled microarray to determine the identity and quantity of labeling to the various mRNA probe sites and thus providing an expression profile of small RNA.

### RELATED APPLICATIONS

This application is a continuation-in-part of PCT 2006003977, filed Feb. 6, 2006, and U.S. application Ser. No. 11/116,935, filed Apr. 28, 2005, which claims the benefit of Provisional Application No. 60/650,034, filed Feb. 4, 2005.

### TECHNICAL FIELD

The present invention relates generally to methods of analyzing small RNAs including microRNAs and siRNAs, and whole-genome transcripts for all coding and non-coding transcripts.

### BACKGROUND

The completion of Human Genome Project and genome sequencing on other species has had an enormous impact on human healthcare, quality of life, food, environment, and other living organisms. Sequencing of many other species is continuing. Now the major task is to understand the function and products of genomes. For a long time, there was a belief that RNA function was limited to heir involvement in protein synthesis including messenger RNA, ribosomal RNA and transfer RNA (mRNA, rRNA, and tRNA, respectively). Recently identified small non-coding RNA molecules were discovered to be actively involved in gene regulation by directly interacting with mRNAs and silencing the genes, commonly referred to as RNA interference (RNAi), and play an important role in biological pathological processes of cells. These RNA molecules are further classified into microRNAs (miRNAs) and small interfering RNAs (siRNAs) and so on.

The miRNAs and siRNAs are classes of small non-coding RNA molecules that are widely expressed in many cells throughout all eukaryotes. The mature form, or the functional portion of these RNAs is generally small (˜22 nucleotides), the precursors of which are longer, hairpin, single- or double-stranded RNA molecules transcribed from the genome. The complete (as transcribed molecule) is called primary miRNA, or pri-miRNA, and is a large complex oligonucleotide composed of a series of hair-pin loops held together at their base by short single strands of RNA. The pri-miRNA is next shorn of its hair-pin loops, each of which is then called pre-miRNA. The short miRNA is carved out of the stem of the hairpin loop. Other long non-coding transcripts have also been identified, and most of whose functions are unclear. Expression analysis of these miRNA, siRNA and other types of non-coding transcripts in tissues and cells would be greatly helpful for understanding the functions of those molecules and for discovery of biomarkers for diagnostics and therapeutics.

Effective techniques for the detection and quantitation of the small and non-coding RNA expression is lacking. To date, the principal methods used for quantitation of small RNAs are based on gel electrophoresis. The sensitivity is generally low. Cloning and sequencing small RNAs both are time consuming and laborious.

Microarrays have been a powerful tool to profile gene expression, particularly successfully used for mRNA profiling (e.g. GeneChips from Affymetrix and CodeLink from GE Healthcare). However, their use has been limited to mRNA and DNA. Moreover, it becoming appreciated that not just mRNA may be dysfunctional in disease, other species of RNA also may be abnormal. Thus, it would be important to be able to analyze all transcripts of the genome (the transcriptome) in one technique. So far, the procedures of labeling and profiling small RNAs have been quite different since their molecular structure is distinct from mRNAs and DNA. This invention describes a method of labeling and profiling small RNAs as well as the whole-genome transcripts combining all non-capped and capped RNA molecules. The method provides a way to analyze the whole transcriptome at one time and can be applied to microarrays and other detection technologies as well.

### SUMMARY OF THE INVENTION

Herein is described a method of profiling small RNA expression using microarrays. A new, specific method has been developed to perform this application. In this disclosure, miRNAs were used as an example for the method and application. Other types of RNAs can also be analyzed using variations on the disclosed method.

In one embodiment, there is a method of selectively labeling non-messenger RNA molecules by isolating total RNA from a tissue or cell, dissolving the isolated RNA, blocking 3′ ends of the RNA and adding T4 RNA ligase and a labeled nucleic acid adaptor, with the result that the T4 RNA ligase ligates the adaptor only to RNA having a 5′ phosphate group and preferably small RNA are labeled.

In another embodiment, there is a method of selectively labeling non-messenger RNA molecules by isolating total RNA from a tissue or cell, dissolving the isolated RNA, and adding T4 RNA ligase and a labeled nucleic acid adaptor, with the result that the T4 RNA ligase ligates the adaptor to RNA having a 5′ phosphate group. Optionally, the isolated RNA is dissolved in RNase-free water. There can be an additional step of separating small RNA from larger RNA, optionally on a gel or column. The labeled nucleic acid adaptor can be an oligonucleotide with interspersed label. The label may be biotin, a radioactive compound, a phosphorescent compound, or a fluorogenic compound. The biotin labeling is followed by treatment with streptavidin. The streptavidin can be streptavidin Alexa 647. The miRNA or small RNA microarrays so produced can be used in genomic research, drug target validation, drug discovery, diagnostic biomarker identification or therapeutic assessment.

In another embodiment, there is disclosed a method of labeling the 5 end of mRNA that isolates total RNA from a tissue or cell, dissolves the isolated RNA, removes a 5′ cap structure from the mRNA using tobacco acid pyrophosphatase (TAP), and removes the TAP; optionally blocks 3′ ends of the RNA molecules; and ligates an adaptor to the RNA by adding T4 RNA ligase and a labeled DNA or RNA adaptor. Optionally, the isolated RNA is dissolved in RNase-free water. Moreover, the small RNA can be separated from the larger RNA. The small RNA can be separated from the larger RNA on a gel or column. The labeled nucleic acid adaptor can be an oligonucleotide with interspersed label. The label can be biotin, a radioactive compound, a phosphorescent compound, or a fluorogenic compound. When the biotin labeling is used, it is followed by treatment with streptavidin. The streptavidin can be streptavidin Alexa 647. The 3′ end of the RNA can be optionally blocked with deoxynucleotidyltransferase (TdT) and dideoxynucleotide adenine (ddA). The miRNA or small RNA microarrays so produced can be used in genomic research, drug target validation, drug discovery, diagnostic biomarker identification or therapeutic assessment.

In another embodiment, there is disclosed a method of expression profiling small RNA by providing labeled small RNA, providing a microarray comprising a plurality of probes hybridizable to small RNA, incubating the labeled small RNA with the microarray, washing unhybridized RNA from the microarray and drying the microarray, processing post-hybridization RNA on the microarray, and scanning the labeled microarray to determine the identity and quantity of labeling to the various miRNA probe sites and thus providing an expression profile of small RNA. The miRNA or small RNA microarrays so produced can be used in genomic research, drug target validation, drug discovery, diagnostic biomarker identification or therapeutic assessment.

In another embodiment, a method of directly labeling unamplified small RNA targets or non-capped RNA has the steps of a. isolating total RNA; b. dissolving the isolated RNA in H₂O; and c. ligating a labeled adaptor to the uncapped 5′ end of RNA molecules using T4 RNA ligase.

In another embodiment, a method of labeling unamplified mRNA (capped) or a whole transcriptome has the steps of a. isolating total RNA; b. dissolving the isolated RNA in H₂O; c. optionally blocking the 3′ end of target RNA using TdT and ddT; d. de-capping the RNA using TAP; and e. ligating a labeled adaptor to 5′ ends of RNA molecules using T4 RNA ligase.

In another embodiment, a method of labeling amplified RNA targets on small RNAs by sense strand labeling has the steps of a. isolating total RNA; b. dissolving the isolated RNA in H₂O; c. extending 3′ ends of RNA using polyA polymerase and ATPs; d. optionally blocking the 3′ ends using Tdt and ddA; c. ligating a labeled adaptor containing T7 promoter sequence at the S′ portion of adaptor to the 5′ end of RNA using T4 RNA ligase; f. performing reverse transcription using poly T primer, g. filling the full cDNA length using DNA polymerase; and h. performing in vitro transcription using RNA polymerase and labeling the transcripts with labeled nucleotides.

In another embodiments a method of labeling amplified RNA targets on small RNAs by antisense strand labeling has the steps of a. isolating total RNA; b. dissolving the isolated RNA in H₂O; c. extending 3′ ends of RNA using polyA tailing polymerase; d. optionally blocking the 3′ ends of RNA using TdT and ddA; e. ligating an adaptor to the 5′ end of RNA using T4 RNA ligase; f. performing reverse transcription using T7-poly T primer with T7 at the 5′ end to generate the first strand; gf generating the second strand using DNA polymerase and RNaseH; and h. performing in vitro transcription using RNA polymerase and labeling the transcripts using labeled nucleotides.

In another embodiment, a method of labeling amplified RNA targets in the RNA transcriptome has the steps of a. isolating total RNA; b. dissolving the isolated RNA in H₂O; c. de-capping using TAP; d. extending 3′ ends of RNA using polyA polymerase and ATPs; e. optionally blocking the 3′ ends using TdT with ddA, ddG, ddT, ddC, or a combination thereof; f. ligating an adaptor containing T7 promoter sequence at the 3′ portion of adaptor to 5′ ends of RNA using T4 RNA ligase; g. performing reverse transcription using polyT primer; h. filling the full cDNA length using DNA polymerase; and i. performing in vitro transcription using RNA polymerase and labeling the transcripts with labeled nucleotides.

In yet another embodiment a method of labeling amplified whole-genome transcripts by antisense strand labeling has the steps of a. isolating total RNA; b. dissolving the isolated RNA in H₂O; c. decapping RNA with TAP; d. extending 3′ ends of RNA using polyA; e. optionally blocking the 3′ end of RNA using TdT with ddA, ddT, ddG, ddC, or a combination thereof; f. ligating an adaptor to S′ ends of RNA using T4 RNA ligase; g. performing reverse transcription using T7-polyT primer with T7 at the 5′ ends to generate the first strand of DNA; h. generating second strands of DNA using DNA polymerase and RNaseH; and i. performing in vitro transcription using RNA polymerase and labeling RNA transcripts using labeled nucleotides.

### BRIEF DESCRIPTION OF THE DRAWINGS

FIG. 1 includes schematic depictions of the inventive methods of unamplified and RNA direct labeling. Line 1 shows small RNA (i) and mRNA (ii). After 3′ end blocking, the second line shows both types of RNA are blocked (x at the 3′ end). These blocked RNAs are then added to a labeled adaptor (B—OH), as shown in line 3. In line 4 only the small RNA and RNA with a phosphate at the 5′ end can be ligated to the adaptor; there is no ligation of the other mRNA species. If decap (TAP) is added, then whole-genome transcripts can be labeled. FIG. 1B is a schematic depiction of the inventive method of labeling selectively amplified genome transcripts or labeling the amplified whole-genome for both sense and antisense strands. The left or i shows sense strand RNA labeling. The 3′ end of the RNA is extended by polyA tailing, the 3′ end is blocked. A T7 adaptor is ligated to the 5′ end. Reverse transcription is performed using a polyT primer. RNA is generated by in vitro transcription while the RNA are labeled using e.g., biotin. For mRNA, the additional step of decapping is performed. The right figure of ii shows antisense strand RNA labeling. In addition to the preceding steps, there is second synthesis of a second strand of DNA prior to in vitro transcription of the RNA.

FIGS. 2A and 2B are examples of miRNAs profiled on microarrays. Total RNA was isolated and labeled with biotinylated adaptors as outlined in FIG. 1. The labeled RNA samples were processed on microarrays for hybridization. The arrays were scanned and the images of two tissue samples shown above were for human lymphoma (2A) and placenta (2B). The different patterns in FIGS. 2A and 2B illustrate differential expression of miRNAs for the two exemplary tissues.

FIG. 3 is a graph derived from a Northern blot assay for confirmation of microarray information. Five miRNA probes (let-7b, miR-1b, miR-100, miR-125b and miR-128) were chosen for use in the Northern blot Expression levels were normalized log values taken for comparison plotting. A significant: correlation (p<0.05) was observed with r=0.83.

FIG. 4 is a bar graph showing results with (solid bars) and without (bars with diagonal lines) RNA fractionation, a method commonly used to study small RNA. Total RNA was first isolated using the Trizol method. Then RNA was either fractionated using size selection columns or was not fractionated. No significant differences were observed in the signal numbers and intensity between the paired columns.

FIG. 5 shows the image of a microarray of the gene expression of mRNA and miRNA representing the whole-genome profiling. The antisense method of FIG. 1.B.ii. was used.

FIGS. 6a and 6b show a side-by-side comparison of microarrays produced by the blocking (FIG. 6a) and no-blocking (FIG. 6b) methods. No significant difference was discernible.

FIG. 7 is a signal intensity plot graph. No significant difference between the two sets of signals was observed (p=0.6).

### DETAILED DESCRIPTION

This invention provides a method to profile small RNA and whole-genome transcripts in cells, tissues and organisms. The specific technical platform used herein was microarrays, because microarrays afford a powerful system for massive and parallel detection of enormous numbers of RNA targets at the same time. Microarrays have become a common tool for functional genomic research and molecular profiling. We show their use with new methods of generating RNA transcripts.

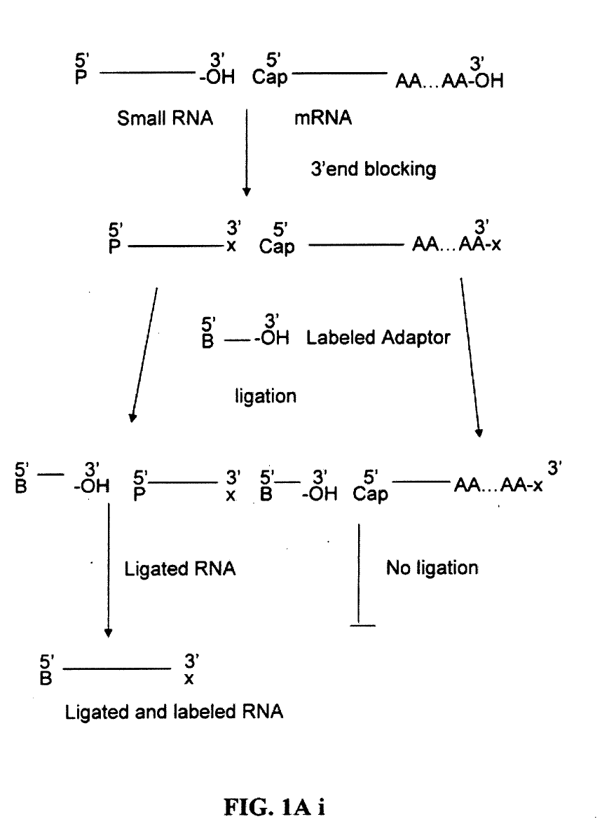

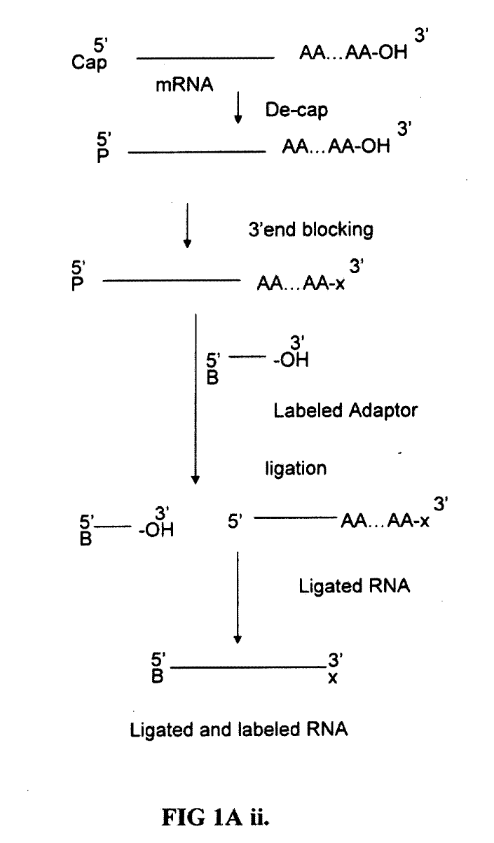

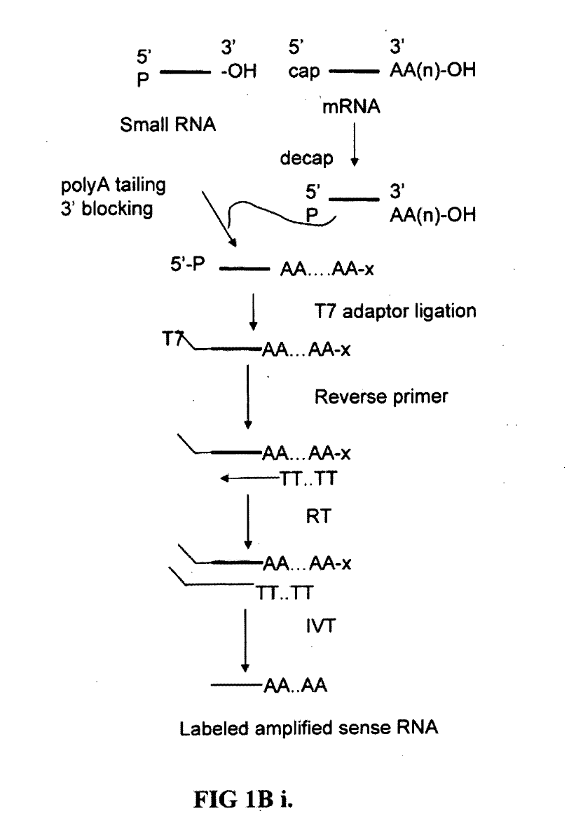

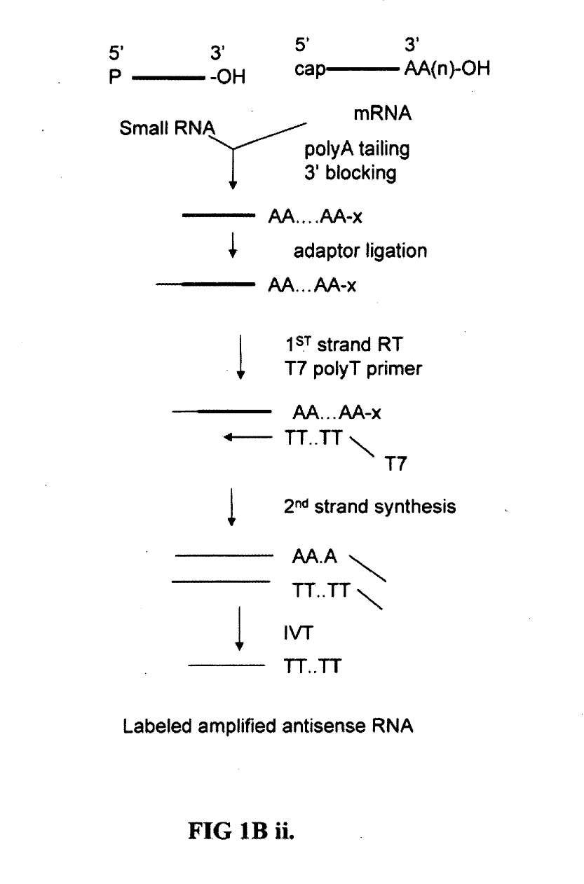

MicroRNAs, siRNAs, and other small non-coding RNAs, unlike mRNAs, have a phosphate group at their 5′ end and no 5′ structural modification (or 5′ cap). This phosphate provides a functional group to covalently bond to an —OH group at the 3′ end of an oligonucleotide adaptor. RNA ligase connects two molecules between the 5′ phosphate group on the RNA molecule and 3′-OH group of adaptors (FIG. 1). This method provides many advantages.

The first and foremost advantage of this method is selective labeling. All RNA molecules have 3′-OH groups. Whereas, two types of structures are identified at the 5′ ends of RNA: the 5′ cap for mRNA and a 5′ phosphate group for small RNA and other non-coding RNA molecules. Direct ligation only labels non-capped RNA molecules including small RNAs, thus providing a means for selective labeling and reducing labeled species complexity. If mRNAs need to be ligated with adaptors, a decap step is then required.

The second advantage owes its benefit to the fact that T4 RNA ligase can ligate RNA to single-stranded DNA/RNA oligonucleotide adaptors. Adaptors can be DNA or RNA or mixtured modified and labeled with different molecules, including but not limited to biotin, radioactive, fluorescent, and energy transfer compounds.

To avoid sample RNA inter- and intramolecular interaction, the 3′-OH of RNA must be blocked before ligation. Herein were used dideoxynucleotide adenine (ddA) and terminal deoxynucleotidyltransferase (TdT) to block all 3′-OH groups of RNA; other hydroxyl blockers are well known in the art and include but are not limited to ddT, ddG and ddC. In a variation of the method, no blocking was performed.

Third, this method of labeling ensures a good labeling ratio and efficiency between RNA targets and adaptors, reducing the differential and bias labeling that most chemical, UV cross-linking, and 3′ extension methods create.

Microarrays generally consist of a substrate matrix and nucleotide probes. Organic polymers or treated glass are used for a matrix that is supportive and biocompatible. Frequently, polymer chemistry modifications are used for better nucleotide probe attachment. The attached nucleotide probes are designed to hybridize target sequences. The attached nucleotide probes that are complementary to labeled RNA targets are printed or otherwise spotted on the matrix and immobilized on the arrays. Nucleotide probes can also be photolithosynthesized in situ on the arrays.

DNA probes on the microarrays were used in the experiments for miRNA and mRNA detection. The probes were designed for optimal temperature/salt concentrations as well as hybridization properties. The probes were printed on the arrays. To determine whether the microarray technique were robust, 210 probes representing miRNAs and mRNAs were designed, printed on the microarrays and tested with good results.

As used herein, the terms “complementary” or “complementarity” are used in reference to polynucleotides (i.e. a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in detection methods that depend upon binding between nucleic acids. Either term may also be used in reference to individual nucleotides, especially within the context of oligonucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

The detected materials are RNA that has been isolated from cells or tissues. The invention does not depend on the method of harvesting the RNA. Any accepted method for harvesting RNA can be used, including but not limited to, phenol/chloroform separation, as well as commercial kits and columns, such as the TRIZOL reagent (Invitrogen). The harvested RNA that is detected can be, but need not be, total RNA or size-selected RNA.

Subsequently the harvested RNA was labeled for further analysis. Total RNA, fractionated RNA, or other types of treated RNAs can be labeled according to the same principle. The label is part of a signaling oligonucleotide or single nucleotide that chemically combines with the RNA.

A variety of signals can be incorporated into the signaling oligonucleotide. These include, but are not limited to, radioactive compounds (including but not limited to P³², P³³, S³⁵, I¹²⁵ and I¹³¹ or others known in the art or discovered in the future). Additional alternative labels include, but are not limited to, dyes; binding moieties such as biotin; haptens such as digoxigenin; luminogenic, phosphorescent or fluorogenic moieties; mass tags; and fluorescent dyes alone or in combination with moieties that can suppress or shift emission spectra by fluorescence resonance energy transfer (FRET); and enzymatic substrates. Labels may provide signals detectable by fluorescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or alternatively, may be charge neutral. Adaptors can include or consist of nucleic acid or protein sequences, so long as the sequence comprising the label is detectable.

Certain nucleotide bases not commonly found in natural nucleic acids may be included in the nucleic acid adaptor or in the probe attached to the microarrays of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

In addition, “nucleotide analogs” as used herein refers to modified or non-naturally occurring nucleotides including, but not limited to, analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242; B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having modification on the sugar moiety, such as dideoxy nucleotides and 2′-O-methyl nucleotides. Nucleotide analogs include modified forms of deoxyribo-nucleotides as well as ribonucleotides.

Biological samples may be animal, including human (normal or abnormal), fluid, solid (e.g., stool) or tissue, as well as liquid and solid food and feed products and ingredients such as dairy items, vegetables, meat and meat by-products, and waste. Biological samples may be obtained from eukaryotic organisms such as yeasts, plants and animals, including but not limited to all of the various families of domestic animals, as well as feral or wild animals, including, but not limited to, such animals as birds, ungulates, bear, fish, lagomorphs, rodents, etc. Fungi, yeasts, bacteria, viruses and prions also can be sampled.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

This inventive method of labeling small RNA can be used in genomic research, drug target validation, drug discovery, diagnostic biomarker identification or therapeutic assessment.

RNA can be isolated by any methodologies mentioned above. Isolated total RNA was dissolved in RNase-free water for the labeling protocol described below.

Profiling methodology described herein was microarray biochips. Probes on the microarray are designed complementary to the labeled RNA. Probes include sequences complementary to small non-coding RNAs and coding mRNA. The selection and design are based on their specificity, position on the genes, GC content, Tₘ, hairpin structure, length, etc. MicroRNA and siRNA probes were designed based on their processing structure in the biogenesis pathways. For instance, they have mature forms, precursors, and counterparts (miR*). The mature forms are usually about 22 nt long, the same as the counterparts. The precursors vary a lot depending on the stages of biogenesis. Pre-miRNAs are 60-110 nt and pri-miRNAs are about 1 kb or even longer. The probes are selected based on their structure for specificity and unique sequences. For example, mature probes are designed based on the mature sequences, counterparts (miR*) on the counterpart sequences, pre-miRNAs on the loop portion, and pri-miRNAs on the region beyond the pre-miRNA sequences. The probes designed in this study were DNA oligos with 30 nt long, computed with the designated algorism, and printed on the microarray slides using OmniGrid Arrayers (Genomic Solutions Inc.). Labeled RNA hybridizes with probes on the arrays. The signal intensity of all probes was scanned and analyzed.

In this invention, RNA labeling technology is divided into two categories: direct labeling on unamplified target RNAs and labeling on amplified target RNAs. For the unamplified RNA targets, directly labeling of an adaptor to non-capped small RNA is achieved using T4 RNA ligase (FIG. 1A-i). If examination of mRNAs is needed, the 5′ cap can be removed from the mRNA. The decapitating (or decapping) step can be inserted before line 1 or between lines 1 and 2 in FIG. 1A-ii. For amplified RNA, two strategies were used: sense strand RNA and antisense strand RNA. The common procedure is to extend 3′ by poly (A) tailing using poly (A) polymerase, optionally block the 3′, ligate an adaptor to the 5′ end so that all RNA molecules have a polyA tail at the 3′ end, and attach an adaptor at the 5′ end. The new form of RNA is then reverse transcribed by polyT primers to form first- (sense) and second-strand DNA. Using T7 RNA polymerase, an in vitro transcription assay is performed to generate and amplify labeled RNA molecules. The only difference between sense and antisense strand RNA is the position of T7 promoter. For the sense RNA, the T7 promoter is in the adaptor for 5′ ligation; for antisense, the T7 promoter is in the polyT primer (FIG. 1B).

In summary, the experimental protocols are described below. For the direct labeling, the 3′-OH of RNA was optionally blocked by dideoxynucleotide adenine (ddA; Amersham) using terminal deoxynucleotidyltransferase (TdT; New England Biolab). Instead of ddA, ddT, ddG or ddC may be used. Even without 3 end of the RNA being blocked, the RNA are molecularly ligated to a labeled adaptor, such as 5′ biotin-AAAAAAAAAAAAAAA-biotin-AAAAA-3′. Other natural nucleotides as well as synthetic nucleotides can be used, as discussed above. The length of the oligonucleotide can be varied from 1 to hundreds, as can the number of signaling compounds in the labeled adaptor, for example, from one to ten, 20, 30, 40 or more. Those skilled in the art can optimize the oligo adaptor length as well as the signaling moiety. Signaling moiety size is limited by cost and/or quenching and other factors. To label the small RNA, the adaptors and T4 RNA ligase were added to the optionally blocked RNA solution to produce the labeled RNA.

The hybridization conditions and temperature typically depend on the types of microarrays and length of array probes. In these miRNAs experiments, the microarray probes were about 30 nt long, and sequences were designed based on and complementary to known miRNA sequences. With added formamide and SSPE (or SSC) buffer, the hybridization on the array chips took place overnight. Timing and temperature can be optimized by those of skilled in the art, after seeing the following examples. After incubation, the arrays were washed and stained with a variety of dyes, depending on the adaptor label. With biotin, the form of streptavidin (Alexa 647 from Molecular Probes) is preferred. After repeatedly washing and then drying, the microarrays were scanned, the signal intensity of all probes was recorded and computed. The miRNA expression profiling for the samples were analyzed and compared with other microarray results.

For amplified sense stranded RNA, RNA at the 3′ end was extended by tailing polyA using polyA polymerase (Epicentre). The 3′ end was blocked and an adaptor was ligated described above. The adaptor sequence composed T7 promoter TATACGACTCACTATAGGCGATCGTCACCGTGTAGAGA. The new form RNA was then used for reverse transcription for the first strand cDNA using polyT primer. After DNA polymerase and RnaseH, in vitro transcription (IVT) was performed to generate sense labeled RNA. The labeling molecule used was biotin-ATP. For amplified antisense RNA, the protocol was similar to the above, except the adaptor had no T7 promoter sequence. Instead, the T7 polyT primer was at the 5′ end. Thus, antisense RNA was generated with biotin-UTP as labeling molecules.

Following are examples using total and fractionated RNA.

### EXAMPLE 1

This experiment includes total RNA isolation, RNA labeling, microarray hybridization, array scanning and data analysis for profiling. See FIG. 1 for detailed steps.

Total RNA was isolated from 8 tissues or cells (liver, kidney, lungs, placenta, testis, prostate and lymphoma) using the phenol and chloroform method, or TRIZOL reagent (Invitrogen). The obtained RNA was dissolved in RNase-free water. Five micrograms of total RNA was used. If total RNA is used, it is preferable to use about 10 μg; whereas, if fractionated miRNA is used, amounts as low as 1 μg can be used.

3′ end blocking. 10 μg of total RNA, 1 nmol ddA 50 mM K Acetate (Ac), 20 mM Tris buffer with acetate (pH 7.9), 10 mM MgAc₂, 1 mM dtt, 0.25 mM CoCl₂, 6.7% DMSO (Sigma), 4 units of TdT in a final volume of 15 μl. One nmol dideoxynucleotide adenine (ddA) and 4 units of terminal deoxynucleotidyl-transferase (TdT) were added in 5 μg of total RNA samples to make a volume of 10 μl. The reaction was performed at 37° C. for an hour, then heated up to 70° C. for 20 minutes to inactivate the TdT. Thus, the 3′-OH groups were blocked by attachment of ddA. This step prevents miRNA intermolecular ligation at the next step.

Ligation. A biotin-labeled DNA adaptor was used for the ligation. 500 μmol adaptors (5′ biotin-AAAAAAAAAAAAAAA-biotin-AAAAA-3′) and 5 units of T4 RNA ligase (New England Bioscience) were added to the previously blocked RNA solution. In addition, a ligation buffer was added to a final concentration of 40 mM tris-Ac (pH 7.9), 37 mM KAc, 10 mM MgCl₂, 3.5 mM DTT, 0.25 mM ATP, 0.18 mM CoCl₂, 10% DMSO and 5 mg/mL bovine serum albumin (BSA). The reaction was performed at 37° C. for 2 hr (although the temperature and time can be varied). Then the solution was heated and maintained at 65° C. for 15 min to inactivate the ligase.

Storage or microarray hybridization. Labeled RNA samples can be stored frozen at −70° C. (or lower) or directly applied to the microarray. The hybridization conditions and temperatures depend on the types of microarrays and the length(s) of array probes. In this miRNA case, the miRNA probes were 30 nt long and sequences were designed to be complementary to the miRNA sequences. With 30% formamide (can be varied between 10 and 50%) and 6×SSPE buffer (optionally SSC), the hybridization on the array chips was maintained at 30° C. for 16 hr or overnight.

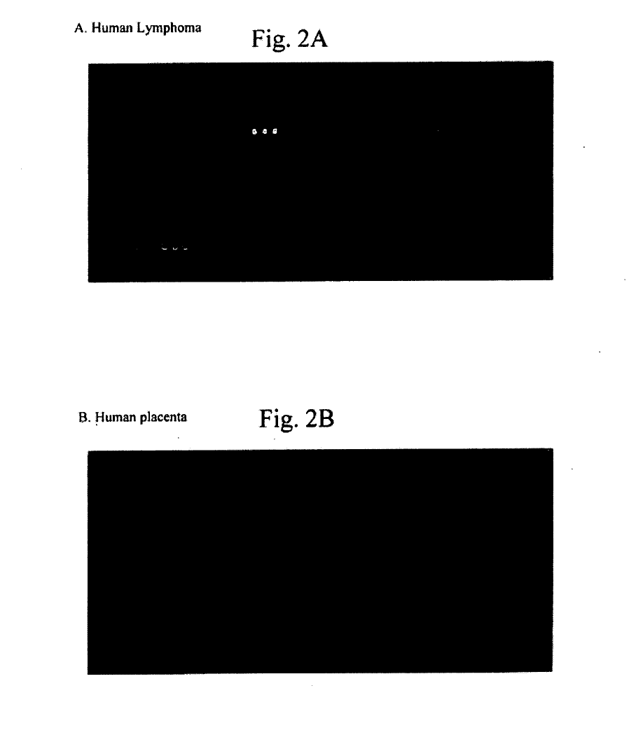

The arrays were washed and stained with streptavidin-Alexa dyes (Genisphere), Customary buffers were used for washing, including 1×SSC with 0.1% SDS, and then 0.2×SSC at 37° C. Final washes were in water. After the arrays dried, they were scanned by a scanner. The signal intensities of all probes on the arrays were recorded and computed. The miRNA expression profilings for the samples were analyzed. The results for lymphoma and placenta are shown as FIG. 2A and FIG. 2B, respectively. The patterns of miRNAs in these tissues were markedly different.

### EXAMPLE 2

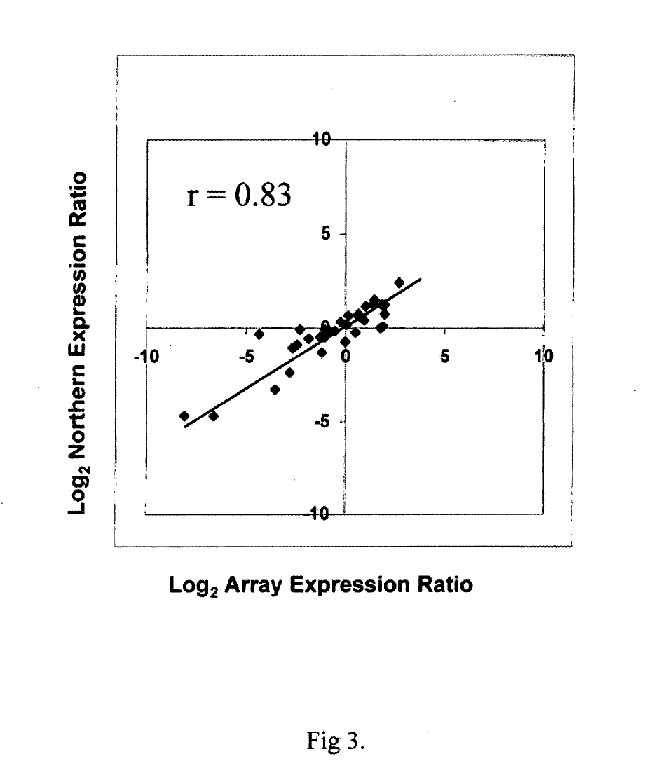

Five miRNAs were selected for the comparison of this inventive labeling method on microarrays with the standard Northern blot method. The DNA probes (the same sequences as on the arrays) used for the Northern were radioactively labeled (P³³) and the Northern method was performed on eight different human tissues. The result shows a significant correlation (p<0.05) between the array results using the described method on microarrays and on Northern blot (FIG. 3).

### EXAMPLE 3

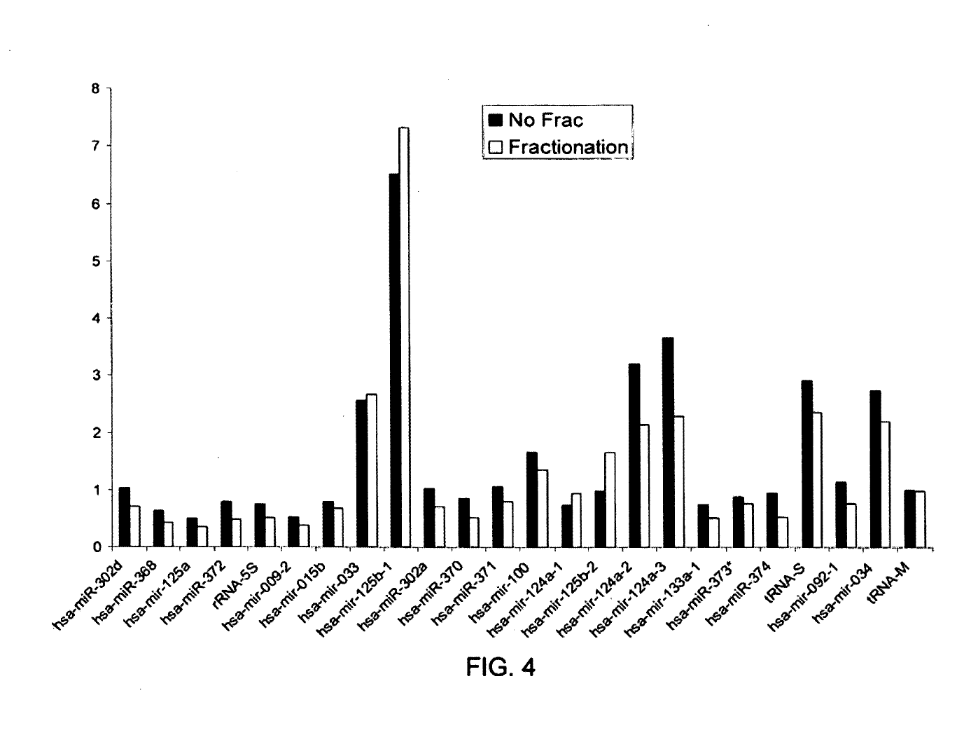

Fractionated RNA samples were also tested. RNA was isolated from three different cell lines using Invitrogen MyRNA kit, or a similar kit from Ambion, to enrich for small RNA molecules. As described above, only 0.5 μg of fractionated RNA was used for labeling. The labeled RNA was then used for hybridization on the arrays. The results were used to compare with total RNA experiments isolated from the same tissues (FIG. 4). The signal patterns and intensity significantly correlated, suggesting the efficiency of labeling.

### EXAMPLE 4

If examination of 5′ end of mRNAs is needed, then labeling of 5′ end mRNA becomes necessary. The above methodology can be used for this application. One additional step is needed to remove the 5′ cap structure from mRNA. The “decap” step can be inserted between steps 1 and 2, or between steps 2 and 3. To remove the 5′ cap from the mRNA, the mRNA was exposed to 1 unit of tobacco acid pyrophosphatase (TAP) at 37° C. for 1 hr. Followed by phenol/chloroform purification, the decapped samples can be processed as in step 2 or step 3.

### EXAMPLE 5

RNA isolation. Total RNA (5 g) from human liver tissue was isolated for use in the assay.

Poly A tailing. This isolated RNA used for the poly A tailing reaction was in a 50 μl (50 mM Tris-HCl, pH 8.0, 250 mM NaCl, 10 mM MgCl₂, 1 mM ATP) with 8 units of A-Plus PolyA polymerase (Epicentre). The reaction was incubated at 37° C. for 30 min. Alter ethanol precipitation and resuspension in 10 μl, the reaction mixture was ready for 3′ blocking.

Blocking. Next 10 μl tailed RNA was blocked in 15 μl reaction with 4 U TdT (described above) at 37° C. for 1 hour. The enzyme was inactivated by heating up 75° C. for 20 minutes.

Ligation. An adaptor was ligated at the 5′ end of RNAs. The sequence included the T7 promoter sequence for the sense strand RNA amplification, or without the T7 sequence for antisense target amplification. The reaction mixture was 20 μl with 5 U T4 RNA ligase (described above). The reaction was at 37° C. for 2 hr.

Reverse transcription. After ethanol precipitation and resuspension in 6 μl H₂O, the RNA was reverse transcribed using either poly 124 primer for the sense or T7-polyT24 primer. Five μl 100 ng/μl primer was used with 1 μl 10 mM dNTP mix. The reaction was mixed at 65° C. for 5 min and then chilled on ice. Four μl 5× first strand buffer, 2 μl 0.1 M DTT and 200 unit (Invitrogen) reverse transcriptase was incubated at 42° C. for 50 min. DNA polymerase and Rnase H mixed with dNTPs (Invitrogen) were incubated at room temperature for 2 hr.

IVT. After ethanol precipitation and resuspended in 8.45 μl H₂O, the in vitro transcription assay was performed. RNA polymerase mixed with buffered NTPs and biotin-ATP or biotin-UTP was reacted at 37° C. for 16 hr.

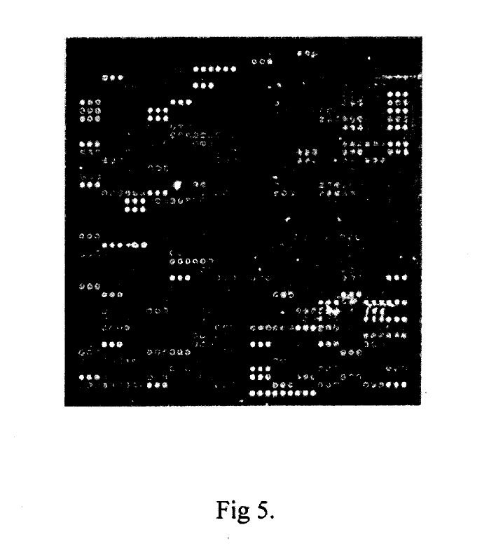

After ethanol precipitation and resuspension in 49 μl H₂O, 40 μl of the solution was used for hybridization and the rest for quantification. Hybridization and microarray processes were described above. Array images are shown on FIG. 5.

### EXAMPLE 6

After the previously described examples were performed, the process was modified to remove the blocking step. Experiments without blocking showed no deterioration in results and a benefit in less time and fewer reagents.

Total RNA was obtained using the TRIZOL method. The obtained RNA was dissolved in RNase-free water. Five micrograms of total RNA was used. Without prior blocking, RNA samples were directly labeled using 5 units of T4 RNA ligase and 500 μmol biotinconjugated adaptor described previously in 40 mM tris-Ac (pH 7.9), 37 mM KAc, 0 mM MgCl₂, 5 mM DTT, 0.25 mM ATP, 0.18 mM CoCl₂, 12% DMSO and 10 mg/mL bovine serum albumin (BSA). The reaction was performed at 37° C. for 3 hr. Then the solution was heated at 65° C. for 15 min to inactivate the enzyme.

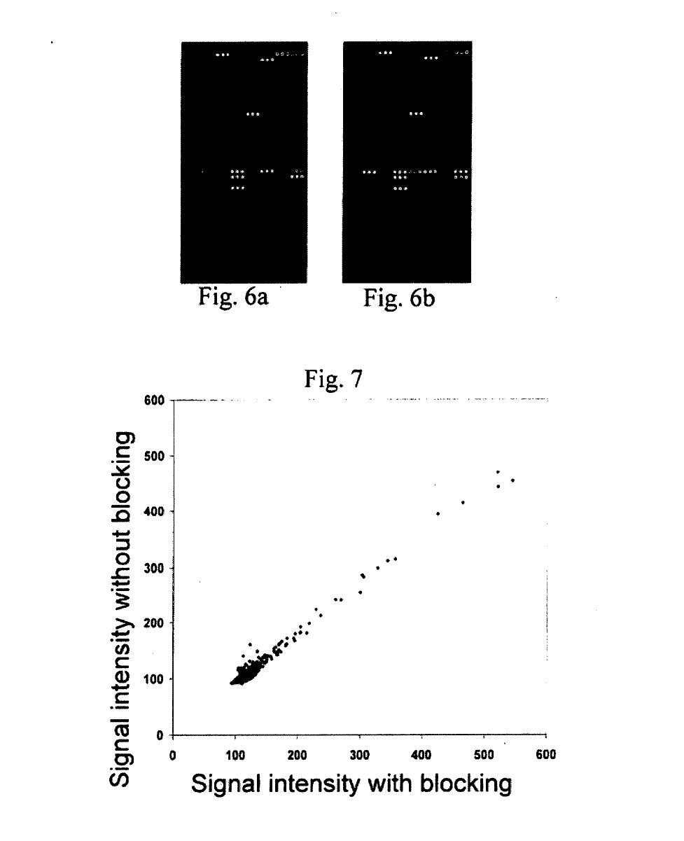

The identically labeled samples, with and without blocking, were hybridized with miRNA probes on microarrays under the same conditions described above. The microarrays of blocked and unblocked samples are displayed side-by-side in FIGS. 6a and 6b and nearly indistinguishable. The signal intensities from the two microarrays are graphed by intensity in FIG. 7. The signal intensities were analyzed and did not differ significantly (p=0.6). Thus, the blocking step that is typically performed to avoid inter-molecular reactions can be omitted with no significant difference. This method is more economical in time and reagents.

The inventions described herein cover new methods of both small RNA (miRNA, siRNA, and non-coding RNA) profiling and whole-genome transcripts (all coding mRNA and all non-coding RNA) profiling on microarrays that composes RNA isolating, labeling and profiling methodologies. The labeling methodology efficiently labels small RNAs and other non-capped RNA molecules. The method also is adaptable to capped RNA molecules (mRNAs). The 5′ end of RNA molecules are labeled using adaptors by ligation and can be applied to the following molecules: microRNAs, siRNAs and other small RNAs, any other RNAs without 5′ caps, and mRNAs after 5′ decapping.

Combining 5′ ligation and 3′ tailing methods, labeling on amplified target RNA is achieved with the IVT. The method of combining 5′ ligation and 3′ tailing allows gene expression profiling not only for small RNAs, miRNAs, siRNAs, and other non-coding RNAs, but also for whole-genome transcripts. For the antisense target approach, decapping is not necessary, which is more advantageous for whole-genome profiling.

This method significantly improves the current microarray gene expression profiling technologies that only analyze mRNA. Due to the rapid expansion of genome information, especially proven actions of miRNAs, siRNAs, and other non-coding RNAs in biological functions, profiling whole-genome transcripts in cells and tissues has become even more important.

This method can be used in many technology platforms, including but not limited to microarray, bead, flow cytometry, and northern blot analyses.

The method will be useful in numerous applications, such as genomic research, drug target validation, drug discovery, diagnostic biomarker identification and therapeutic assessment.

While the foregoing specification discloses how to use and make the invention, the specification is not to be used to define the invention, which is the province of the claims.

1. A method of selectively labeling non-messenger RNA molecules, the method comprising
a. isolating total RNA from a tissue or cell;
b. dissolving the isolated RNA;
c. blocking 3′ ends of the RNA; and
d. adding T4 RNA ligase and a labeled nucleic acid adaptor,
whereby the T4 RNA ligase ligates the adaptor only to RNA having a 5′ phosphate group and only non-capped RNA are labeled.

2. An improved method of selectively labeling non-messenger RNA molecules, the method comprising:
a. isolating total RNA from a tissue or cell;
b. dissolving the isolated RNA; and
c. adding T4 RNA ligase and a labeled nucleic acid adaptor, whereby the T4 RNa ligase ligates the adaptor to RNA having a 5′ phosphate group.

3. The method of claim 2, wherein the isolated RNA is dissolved in RNase-free water.

4. The method of claim 2, wherein after step b, the small RNA are separated from the larger RNA.

5. The method of claim 4, wherein the small RNA are separated from the larger RNA on a gel or column.

6. The method of claim 2, wherein the labeled nucleic acid adaptor comprises an oligonucleotide with interspersed label.

7. The method of claim 4, wherein in step c, the label is selected from biotin, a radioactive compound, a phosphorescent compound, or a fluorogenic compound.

8. The method of claim 7, wherein when the biotin labeling is used, it is followed by treatment with streptavidin.

9. The method of claim 8 wherein the streptavidin comprises streptavidin Alexa 647.

10. The method of claim 2, further comprising using the labeled miRNA or small RNA in genomic research, drug target validation, drug discovery, diagnostic biomarker identification or therapeutic assessment.

11. A method of 5′ labeling mRNA, the method comprising
a. isolating total RNA from a tissue or cell;
b. dissolving the isolated RNA;
c. removing a 5′ cap structure from the mRNA using tobacco acid pyrophosphatase (TAP);
d. removing the TAP; and
e. ligating an adaptor to the 5′ end of the RNA by adding T4 RNA ligase and a labeled DNA or RNA adaptor.

12. The method of claim 11, wherein the isolated RNA is dissolved in RNase-free water.

13. The method of claim 11, wherein after step b, the small RNA are separated from the larger mRNA.

14. The method of claim 13, wherein the small RNA are separated from the larger RNA on a gel or column.

15. The method of claim 11 wherein the labeled nucleic acid adaptor comprises an oligonucleotide with interspersed label.

16. The method of claim 11, wherein after step d and before step e, there is an additional step of blocking the 3′ end of the mRNA molecules.

17. The method of claim 11, wherein in step e, the label is selected from biotin, a radioactive compound, a phosphorescent compound, or a fluorogenic compound.

18. The method of claim 17, wherein when the biotin labeling is used, it is followed by treatment with streptavidin.

19. The method of claim 18 wherein the streptavidin comprises streptavidin Alexa 647.

20. The method of claim 11, further comprising the step of using the miRNA or small RNA in genomic research, drug target validation, drug discovery, diagnostic biomarker identification or therapeutic assessment.

21. A method of expression profiling small RNA, the method comprising
a. providing labeled small RNA from claim 1;
b. providing a microarray comprising a plurality of probes hybridizable to small RNA;
c. incubating the labeled small RNA with the microarray;
d. washing unhybridized RNA from the microarray and drying the microarray;
e. processing post-hybridization microarray; and
f. scanning the labeled microarray to determine the identity and quantity of labeling to the various miRNA probe sites,
thereby providing an expression profile of small RNA.

22. The method of claim 20 wherein the miRNA or small RNA microarrays so produced are used in genomic research, drug target validation, drug discovery, diagnostic biomarker identification or therapeutic assessment.

23. A method of directly labeling unamplified small RNA targets or non-capped RNA, the method comprising
a. isolating total RNA;
b. dissolving the isolated RNA in H₂O:
c. optionally blocking the 3′ end of target RNA using TdT with ddA, ddT, ddG, ddC or a combination thereof; and
d. ligating a labeled adaptor to the uncapped 5′ end of RNA molecules using T4 RNA ligase.

24. A method of labeling unamplified mRNA (capped) or a whole transcriptome, the method comprising
a. isolating total RNA,
b. dissolving the isolated RNA in H₂O;
c. de-capping the RNA using TAP;
d. optionally blocking the 3′ end of target RNA using TdT with ddA, ddT, ddG, ddC or a combination thereof; and
e. ligating a labeled adaptor to 5′ ends of RNA molecules using T4 RNA ligase.

25. A method of labeling amplified RNA targets on small RNAs by sense strand labeling, the method comprising
a. isolating total RNA;
b. dissolving the isolated RNA in H₂O;
c. extending 3′ ends of RNA using polyA polymerase and ATPs;
d. optionally blocking the 3′ ends using TdT with ddA, ddT, ddG, ddC or a combination thereof; and
e. ligating a labeled adaptor containing T7 promoter sequence at the 5′ portion of adaptor to the 5′ end of RNA using T4 RNA ligase.
f. performing reverse transcription using poly T primer;
9. filling the full cDNA length using DNA polymerase; and
h. performing in vitro transcription using RNA polymerase and labeling the transcripts with labeled nucleotides.

26. A method of labeling amplified RNA targets on small RNAs by antisense strand labeling, the method comprising
a. isolating total RNA;
b. dissolving the isolated RNA in H₂O;
c. extending 3′ ends of RNA using polyA tailing polymerase;
d. optionally blocking the 3′ ends using TdT with ddA, ddT, ddG, ddC or a combination thereof;
e. ligating an adaptor to the 5′ end of RNA using T4 RNA ligase;
f. performing reverse transcription using T7-poly T primer with T7 at the 5′ end to generate the first strand;
g. generating the second strand using DNA polymerase and RNaseH; and
h. performing in vitro transcription using RNA polymerase and labeling the transcripts using labeled nucleotides.

27. A method of labeling amplified RNA targets in the RNA transcriptome, the method comprising
a. isolating total RNA;
b. dissolving the isolated RNA in H₂O;
c. de-capping using TAP;
d. extending 3′ ends of RNA using polyA polymerase and ATPs,
e. optionally blocking the 3′ ends using TdT with ddA, ddT, ddG, ddC or a combination thereof;
f. ligating an adaptor containing T7 promoter sequence at the 3′ portion of adaptor to 5′ ends of RNA using T4 RNA ligase;
g. performing reverse transcription using polyT primer;
h. filling the full cDNA length using DNA polymerase; and
i. performing in vitro transcription using RNA polymerase and labeling the transcripts with labeled nucleotides.

28. A method of labeling amplified whole-genome transcripts by antisense strand labeling, the method comprising
a. isolating total RNA;
b. dissolving the isolated RNA in H₂O;
c. decapping RNA with TAP;
d. extending 3′ ends of RNA using polyA;
e. optionally blocking the 3′ ends using TdT with ddA ddT, ddG, ddC or a combination thereof;
f. ligating an adaptor to 5′ ends of RNA using T4 RNA ligase;
g. performing reverse transcription using T7-polyT primer with T7 at the 5 ends to generate the first strand of DNA;
h. generating second strands of DNA using DNA polymerase and RNaseH; and
i. performing in vitro transcription using RNA polymerase and labeling RNA transcripts using labeled nucleotides.
